(19) United States
(12) Patent Application Publication
(10) Pub. No.: US 2011/0224575 A1
(43) Pub. Date: Sep. 15, 2011

(54) Needle with Helical Grooves Converting Axial Movement to Rotational Movement

(76) Inventors: Oscar R. Carrillo, JR., Attleboro, MA (US); Robert B. DeVries, Northborough, MA (US); John Golden, Norton, MA (US); John A. Griego, Blackstone, MA (US); William J. Shaw, Cambridge, MA (US)

(21) Appl. No.: 13/033,027

(22) Filed: Feb. 23, 2011

Related U.S. Application Data
(60) Provisional application No. 61/312,374, filed on Mar. 10, 2010.

(51) Int. Cl. A61B 10/00; A61M 5/32

(52) U.S. Cl. 600/566; 604/272

(57) ABSTRACT

A device for penetrating tissues within a living body, comprises a shaft extending from a proximal end which, in an operative position, remains outside the body to a distal end which, in the operative position, is within a living body, the shaft comprising a channel extending therethrough from the proximal end to an opening at the distal end. The device also comprises a needle extending within the channel and comprising a tissue penetrating tip. A portion of an outer surface of the needle includes a first structure wrapping therearound and configured to mate with a second structure formed on a corresponding portion of an inner wall of the shaft. The first and second structures mate with one another so that, as the needle is urged axially through the channel, the mating causes the needle to rotate about an axis of the channel.

### PRIORITY CLAIM

This application claims the priority to the U.S. Provisional Application Ser. No. 61/312,374, entitled “NEEDLE WITH HELICAL GROOVES CONVERTING AXIAL MOVEMENT TO ROTATIONAL MOVEMENT” filed Mar. 10, 2010. The specification of the above-identified application is incorporated herewith by reference.

### BACKGROUND

Needle catheters are often employed to inject medication into a body, obtain fluid and/or tissue samples, etc. In these procedures, a needle is advanced to a target tissue site guided using endoscopic vision systems or other imaging techniques (e.g., ultrasound). The needle is then advanced distally from the catheter to penetrate the target location. In biopsy procedures, suction is then applied (e.g., via a syringe) to draw desired sample tissue into the needle. During this process, the needle may be moved back and forth repeatedly to harvest the desired sample, which is drawn through the needle to a proximal end thereof for analysis. In these cases, tissue trauma is increased due to the increased friction between the needle catheter and the skin and intervening tissue.

### SUMMARY OF THE INVENTION

The present invention is directed to a device for penetrating tissues within a living body, comprising a shaft extending from a proximal end which, in an operative position, remains outside the body to a distal end which, in the operative position, is within a living body the shaft comprising a working channel extending therethrough from the proximal end to the distal end comprising an opening. The device also comprises a needle extending within the working channel, the needle comprising a tissue penetrating distal tip, a portion of an outer surface of the needle including a first structure extending along a path wrapping around a portion of a length of the needle, the first structure configured to mate with a second structure formed on a corresponding portion of an inner wall of the shaft, the first and second structures mating with one another so that, as the needle is urged axially through the working channel, the first and second structures cause the needle to rotate about an axis of the working channel.

### BRIEF DESCRIPTION OF THE DRAWINGS

FIG. 1 shows a cross-sectional view of a distal end of a device according to a first embodiment of the present invention;

FIG. 2 shows a cross-sectional view of a distal end of a device according to a second embodiment of the present invention;

FIG. 3 shows a partially cross-sectional view of a proximal end of a device according to a third embodiment of the present invention;

FIG. 4 shows a perspective view of a device according to a fourth embodiment of the present invention; and

FIG. 5 shows a partial cross-sectional view of a distal portion of the device of FIG. 4.

### DETAILED DESCRIPTION

The present invention, which may be further understood with reference to the following description and the appended drawings, relates to a device for rotating a biopsy needle within the body. It is noted that although the exemplary embodiment of the present invention is described with respect to particular procedures within a living body, the description is not meant to limit the application of the invention, which may be employed in any of a number of procedures requiring the insertion of a needle into a living body.

When performing a biopsy procedure, it is often desirable to screw the needle into the target tissue without coring. This motion allows tissue samples to be captured with less need for suction and the consequent drawing of blood and other non-targeted tissues into the needle. This motion, known as juicing, is generally performed by manually rotating the proximal end of the device to transmit the torque along the device shaft to rotate the needle at the distal end. Unfortunately, this rotation causes the shaft of the device to wind up (i.e., twist), reducing the rotation of the distal tip of the needle when compared to the degree of rotation of the proximal handle. Furthermore, rotating the entire needle catheter may cause pain and injury or result in whipping, as those skilled in the art will understand.

Devices and methods according to an exemplary embodiment of the present invention permit rotation of the distal tip of a needle without requiring rotation of the proximal end thereof. Specifically, the user applies to the device only axially directed forces (i.e., forces directed along a longitudinal axis of the device). A portion of this force is converted at the distal end of the device to a rotational force generating a desired rotation of a needle about the axis.

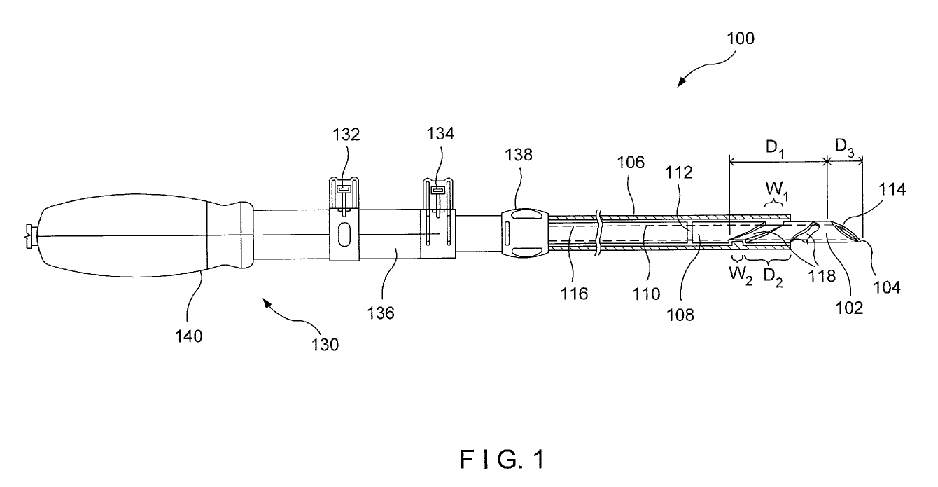

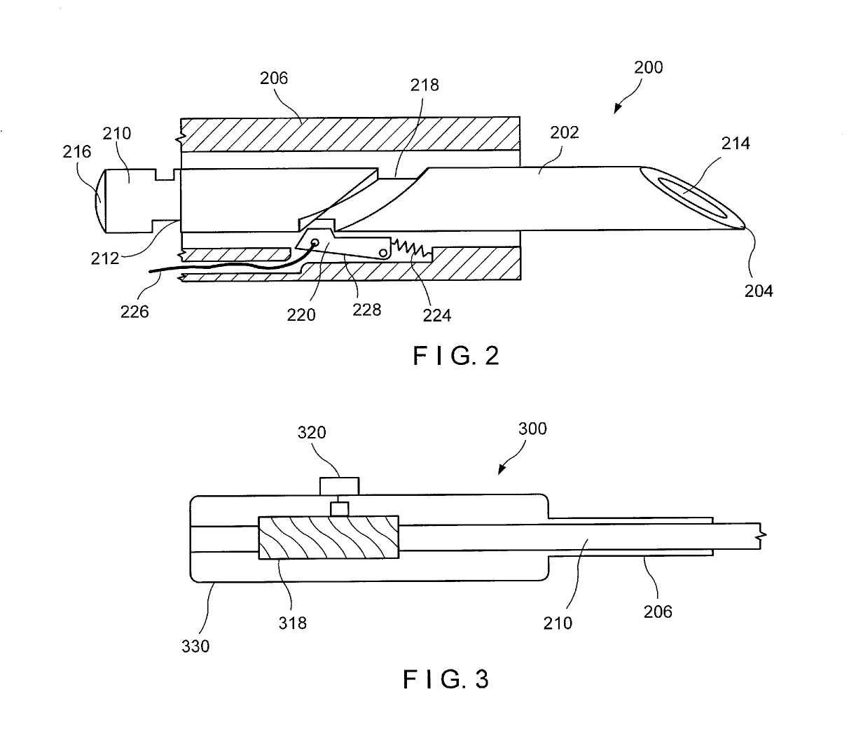

As shown in FIGS. 1 and 2, a device 100 according to a first exemplary embodiment of the present invention includes a needle 102 comprising a puncturing tip 104 at a distal end thereof received within a catheter shaft 106. It is noted that the use of the term distal herein refers to a direction away from a user of the device while the term proximal refers to a direction approaching a user of the device. The proximal portion of the device 100 including a handle 130 remains external to the body accessible to the user while the distal portion, when in an operative position, extends into the body to a target site from which tissue samples are to be obtained.

A distal portion 108 of the needle 102 including the puncturing tip 104 is rotatably attached to a proximal shaft 110 via a rotating joint 112. The joint 112 may allows for any desired degree of rotation between the shaft 110 and the distal portion 108 of the needle 102 and preferably allows for an unlimited range of free rotation. As will be made clear later, any desired limits to the permitted degree of rotation of the distal portion 108 relative to the shaft 110 may be set through the construction of a mechanism for translating axial motion of the shaft 110 into axial and rotational movement of the distal portion 108. However, any desired limit to relative rotation between these parts may also be set through the construction of the joint 112. Furthermore, as would be understood by those skilled in the art, the joint 112 also preferably includes a lumen (not shown) extending therethrough to couple a lumen 114 of the distal portion 108 to a lumen 116 in the shaft 110. The lumen (not shown) thus permits samples to be drawn through the shaft 110 out of the body without removing the device 100 from the body. The lumen of the joint 112 is preferably coupled with the lumens 116 and 114 to provide a smooth unobstructed flow path for materials passed therethrough, thereby preventing any trauma, damage or flow resistance to said materials.

The needle 102 of the device 100 contains a groove 118 extending along a substantially helical path around a portion of an axial length of the needle 102. Specifically, the groove 118 spans a distance D₁ along the needle 102 and terminates at a predetermined distance from the distal end of the needle 102. In an exemplary embodiment, a pitch of the helical groove 118 is selected so that a predetermined amount of axial movement of the needle 102 causes a rotation of approximately 360°, although any other pitch may be employed without deviating from the scope of the present invention. It is noted that although the groove 118 is depicted as extending along only a predetermined distal length of the needle 102, in an alternate embodiment the groove 118 may extend for substantially the entire length of the needle 102. As will be described below in greater detail below, the groove 118 provides for the rotation of the needle 102 upon entry and withdrawal thereof from the catheter shaft 106. A width W₁ of the groove 118 is substantially equal to or slightly greater than a width W₂ of an abutment 120 located on the inner portion of the catheter shaft 106, the groove 118 providing clearance for the sliding of the abutment 120. A height of the abutment 120 relative to a depth of the groove 118 is preferably selected to prevent the removal of the abutment 120 from the groove 118 at any location other than a distal end of the groove 118. During insertion into and removal from the body and when moving within the body between separate target sites, the needle 102 is preferably maintained in a retracted state with the abutment 120 located in a distal most portion of the groove 118 and a pointed distal tip of the needle 102 housed within the catheter 106. Accordingly, a distance D₂ corresponding to an axial distance from a distal end of the abutment 120 to a distal end of the catheter 106 may be substantially equal to or greater than a distance D₃ corresponding to a length of the needle between a distal most portion of the groove 118 and the distal end of the puncturing tip 104. It is noted however, that the distance D₂ may also be smaller than the distance D₃ without deviating from the scope of the present invention. In such an embodiment, the groove 118 may be positioned proximally of the abutment 120 in an insertion configuration and may only be brought into contact with the abutment after the catheter shaft 106 has been brought to a target tissue site in the body so as to prevent trauma to untargeted tissue during insertion, as those skilled in the art will understand. In an alternate embodiment incorporating a cap or other structure to protect surrounding tissue from the puncturing point 104, the distance D₂ may be smaller than the distance D₃.

As the shaft 110 is moved axially in a distal direction via actuation of an actuator on the handle 130 and the needle 102 is extended distally from the catheter shaft 106, the abutment 120 is forced to slide along the groove 118. This causes the needle 102 to rotate (clockwise as viewed looking proximally) as it moves distally.

The abutment 120 also serves to prevent the needle 102 from being extended distally from the catheter 106 beyond a predetermined length. For example, the axial length D₁ of the groove 118 is preferably chosen to permit the needle 102 to protrude distally from the catheter shaft 106 only by a certain distance selected, for example, on the needs of the particular procedure being performed and the mechanical properties of the device 100, as those skilled in the art will understand. After a procedure has been completed, the user of the device 100 retracts the needle 102 by moving the proximal handle portion 140 and the first actuator along at central actuation portion 136 of the handle 130. Specifically, movement of the first actuator 132 in a proximal direction toward the proximal handle portion 140 causes retraction of at least a portion of the device 100 into the handle 130. Movement of the first actuator 132 distally away from the proximal handle portion 140 likewise generates distal movement of the needle 102 until the puncturing tip 104 of the needle 102 is fully exposed outside of the catheter 106. It is noted that the design of the handle 130 may take any desired shape as dictated, for example, by ergonomics, etc. and is not limited to the arrangement shown in the embodiment of FIG. 1. Furthermore, it is noted that the catheter 106 containing the lumen 116, the shaft 110 and the needle 102 may extend proximally from the handle 130 of the device 100 by any length, which length may, for example, be selected to conform to the specific requirements for a procedure being performed. The handle 130 is also configured to permit manual rotation of the device 100 if so desired. Specifically, the catheter 106 may be threadedly engaged with a connector 138 located at the end of the handle 130, the connector 138 being rotatable via manual rotation of a proximal handle portion 140, such rotation being translated only to a lumen (not shown) extending through the handle and to the connector 138 and device 100. Application of this rotational force to the device 100 is likewise converted to rotation of the needle 102, in the opposite direction (i.e., counterclockwise when looking proximally). When the groove 118 of the needle 102 engages the abutment 120, rotation of the needle 102 similarly causes a rotation of the connector 138 and the proximal handle portion 140 until the abutment 120 comes to rest in the distal end of the groove 118 and the puncturing tip 104 fully into the catheter 106.

This exemplary embodiment allows the rate of rotation and the orientation of the puncturing tip 104 of the needle 102 to be controlled to desired rates. Furthermore, as would be understood by those skilled in the art, by varying the pitch or helix angle of the groove 118 along its length, a ratio of the rotational speed of the catheter relative to the axial movement thereof may vary. For example, to ease the start of rotation of the needle 102, the helix angle may be reduced at both the proximal and distal ends of the groove 118 and, then steepen to speed the rotation of the needle as the abutment travels along a middle portion of the groove 118. In addition, since the abutment 120 engages the groove 118 with a substantially tight friction fit, undesired movement of the needle 102 is prevented, improving the accuracy of the movements of needle 102 and eliminating the need for a user of the device 100 to directly apply rotational forces to the proximal end to avoid problems associated therewith (e.g., wind up, whipping, etc.).

As shown in FIG. 2, a device 200 according to an alternate embodiment of the invention is functionally and structurally similar to that of the device 100 except for the differences detailed below. The device 200 comprises a catheter shaft 206 containing a needle 202 with a helical groove 218 formed therein to convert axial movement of a shaft 210 and the needle 202 into a combined rotary and axial motion in the same manner as above in regard to the device of FIG. 1. A lumen 216 in the shaft 210 is fluidly connected via a lumen (not shown) in the rotatable joint portion 212, to a lumen 214 extending through the needle 202. The needle 202 of the device 200 extends from the rotating joint 212 (e.g., a freely rotating joint) at a proximal end to a puncturing tip 204 located at a distal end thereof. The catheter shaft 206 houses therein a pawl 220 which engages with the helical groove 218 in substantially the same manner as the abutment 120 of the device of FIG. 1. Specifically, the pawl 220 engages the helical groove 218 to guide the rotary motion of the needle 202 into and out of the catheter shaft 206 and through the target tissue. The pawl 220 is rotatably coupled to the catheter 206 for movement between an engaged position in which the pawl 220 engages the groove 218 and a retracted position in which the pawl 220 is disengaged from the groove 218 and resides in a recess 228 formed in the wall of the catheter 206. The pawl 220 may, for example, be biased toward the engaged position by a spring 224 or other biasing mechanism as would be understood by those skilled in the art. In addition, a mechanism such as a pull wire 226 or a rod (not shown) coupled to an actuator on the handle (not shown) may be used to pull the pawl 220 into the disengaged position over the bias of the spring 224. This allows an operator to move the pawl 220 between the engaged and retracted positions so that, for example, the needle 202 may be rotated as it is extended and then the pawl 220 may be disengaged to allow the needle 202 to be retracted without rotating. In order to ensure that the pawl 220 is aligned with the groove 218 when the needle 202 is retracted without rotation, the distal end of the groove 218 and the proximal end of the groove 218 must be in substantially the same position around a circumference of the needle 202. In another embodiment of the present invention (not shown), a proximal wall of the pawl 220 may be ramped and the pawl 220 may be seated against the inner wall of the catheter shaft 206 without a recess 228.

As would be understood by those skilled in the art, the aforementioned operation may be automated using known mechanisms. For example, such a mechanism can be coupled to the actuator which applies axial force to the shaft 210 so that, when a user of the device 200 actuates the actuator to withdraw the needle 202 proximally, the pawl 220 is retracted into the recess 228 out of engagement with the groove 218 allowing the needle 202 to withdraw into the catheter shaft 206 without rotating. If the pawl 220 is allowed to spring back against the surface of the needle 202 after being withdrawn from the groove 218, proximal surfaces of portions of the groove 218 circumferentially aligned with the proximal and distal ends of the groove 218 may be ramped to allow the pawl 220 to ratchet therepast as the needle 202 is retracted proximally until the pawl 220 enters and locks into the proximal end of the groove 218. The device 200 thus minimizes rotation of the needle 202 through the tissue reducing trauma thereto.

FIG. 3 shows a proximal portion 300 of a device such as the device 200 permitting a user to selectively engage the pawl 220 with the groove 218 as described above. Specifically, a handle 330 includes a first actuator 318 which applies an axially directed force proximally and distally to the shaft 210 and the needle 202 while a second actuator 320 moves the pawl 220 between the engaged and disengaged positions. This arrangement allows a user to select a rotary needle motion for any portion or a complete duration of a procedure and/or a linear, non-rotational needle movement for any portion or a complete duration of another procedure. As would be understood by those skilled in the art, one or both of the actuators 318, 320 may include any known locking mechanism to maintain the actuator in a desired position.

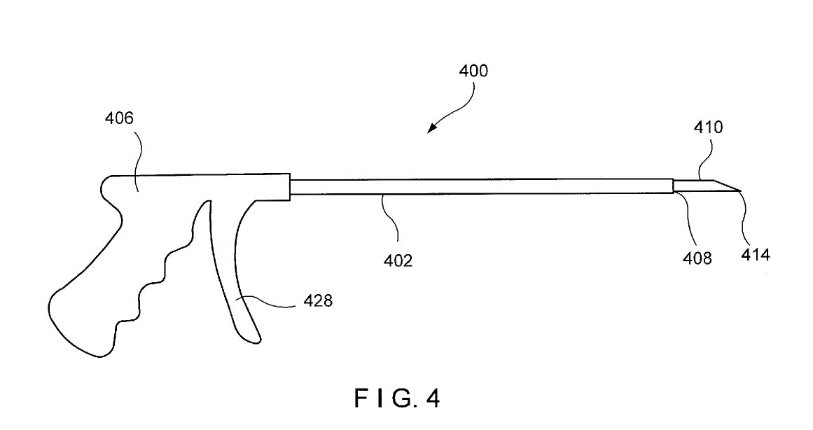

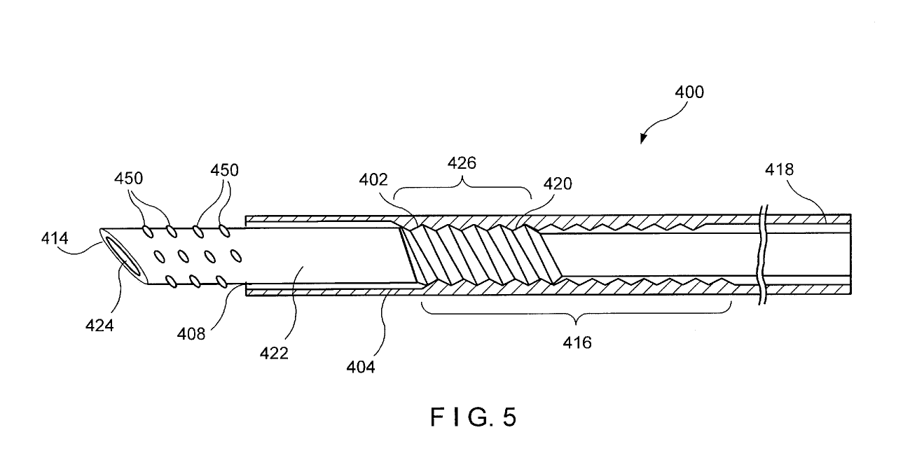

FIGS. 4-5 depict a device 400 according to a fourth exemplary embodiment of the present invention, wherein the device 400 is formed substantially similarly as the device 100 of FIG. 1 with the exception of an advancing mechanism provided thereon and a handle. The device 400 comprises an outer member 402 including a working channel 404 extending from a handle 406 at a proximal end thereof to a distal opening 408. Received within the working channel 404 is a needle member 410 which extends from a proximal end mounted to an actuator 428 of the handle 406 to a tissue penetrating tip 414 at a distal end thereof. A selected portion 416 of the inner walls 418 of the outer member 402 defining the working channel 404 is shaped to correspond to a shape of an outer surface 420 of a corresponding portion of the needle member 410 received therein as will be described in more detail below. The needle member 410 also includes a lumen 422 extending therethrough to a distal opening 424 at the penetrating tip 414. As would be understood by those skilled in the art, an inner surface of the lumen 422 is preferably substantially smooth and free from bumps or ridges which may cause trauma to histological samples drawn thereinto.

As mentioned above, the selected portion 416 of the inner wall 418 is threaded to mate with a correspondingly threaded portion 426 of the needle member 410. The threading engagement of the portion 416 of the inner wall 418 and the portion 426 of the needle member 410 causes the needle member 410 to rotate about its longitudinal axis as it is moved axially through the outer member 402, wherein one or both of the inner wall 418 and the threaded portion 426 are lubricated to better encourage rotation. Since an inner diameter of a portion of the working channel 404 distal of the portion 426 of the needle member 410 is smaller than a maximum diameter of the portion 426, the position of the distal end of the portion 416 defines a maximum extent of projection of the needle member 410 beyond the distal end of the outer member 402. Similarly, since an inner diameter of a portion of the working channel 404 located proximally of the portion 426 of the needle member 410 is smaller than a maximum diameter of the portion 426, the position of the proximal end of the portion 416 defines a maximum extent of withdrawal of the needle member 410 proximally into the working channel 404.

Operation of an actuator 428 on the handle 406 urges the needle member 410 distally and withdraws it proximally substantially along a longitudinal axis of the outer member 402—i.e., axially within the working channel 404. As would be understood by those skilled in the art, the actuator 428 may operate in the manner of any known mechanism for moving a needle axially through a working channel provided the needle member 410 is free to rotate about its longitudinal axis relative to the handle 406. In a preferred embodiment, an actuating mechanism for the needle member 410 may be manually driven by the actuator 428 wherein a reciprocal motion of the actuator 428 is translated to a swiveling proximal-distal movement of the needle member 410. As the needle member 410 moves proximally and distally through the working channel 404, the threaded engagement of the portion 416 of the inner wall 418 and the portion 426 of the needle member 410 causes the needle member 410 to rotate about its axis, causing a corresponding rotation of the distal end of the needle member 410 and the tissue penetrating distal tip 414. This allows the tip 414 to screw into the target tissue without puncturing or coring. This motion allows tissue samples to be captured within the lumen 422 with less need for suction and the consequent drawing of blood and other non-targeted tissues into the lumen 422. This motion substantially mimics a maneuver (juicing) currently performed manually by moving the entire device proximally and distally to drive the tip of a biopsy needle repeatedly into engagement with target tissue. However, the more controlled overall movement and ratio of rotation to linear advancement provided by the device 400 allows for the optimization of this motion to maximize tissue sample quality while minimizing trauma to the surrounding tissue.

A distal portion of the needle member 410 may include a plurality of surface incongruities (e.g., projections 450) extending along a path substantially parallel to a path of the threading of the portion 426 so that, as the needle member 410 is screwed into tissue, the projections 450 rotate along a path substantially similar to that of the portion 426 and, by providing echo-sonic reflection areas when used in conjunction with ultrasound imaging, indicate to a user the precise linear and rotational motion of the distal tip 414 of the needle member 410. The projections 450, in addition to serving as echo-reflectors, also help a user to visualize rotation of the needle member 410 and to provide rotation thereto as the needle member 410 slides against the inner wall 418, the projections being formed of for example, a lubricious polymer such as TFE. Those skilled in the art will understand that the projections 450 are sized to provide a clear image of the motion of the distal end of the needle member 410 but that they preferably project from the outer surface of the needle 426 by a distance which allows the distal tip 414 to be fully withdrawn into the working channel 404 (i.e., the outer diameter of the portion of the needle member 410 including the projections 450 is less than the inner diameter of the distal portion of the working channel 404). In another embodiment of the invention the projections 450 may be formed as any surface features permitting echo-reflection (i.e., raised portions, indentations, insets, etc.). For example, indentations (not shown) provided on the needle member 410 may be formed as one continuous, threadlike spiraling indentation extending around the needle member 410 to permit a screw-like insertion of the needle member 410 into the tissue when coming into contact with a corresponding threading or abutment formed on an outer wall of the working channel 404 or as a plurality of separate indentations spaced from one another to permit an axial insertion into tissue. In another example, insets made of a material with a high echo-reflectivity may be embedded on an outer surface of the needle member 410 to provide a reflective advantage over a solid needle member 410. The insets may be provided in any patterns such as a spiral pattern selected to match a corresponding thread in the working channel 404 and permit a screw-like movement of the needle member 410 thereoutof. The surface features discussed above may be provided from a proximal end located proximally of a needle taper to a distal end of the needle member 410. In a preferred embodiment, the surface features may function as both reflecting agents and as screw guides to permit screwable insertion of the needle member 410 into a target portion of tissue.

The present invention may be applied to medical and scientific procedures that require the insertion of a needle into tissue. Though the present invention has been described with respect to the retrieval of tissue samples, it is submitted that many alternate uses such as, for example, mechanisms according to this invention may be employed for the needles for injection or withdrawal of fluids may be employed without deviating from the spirit and scope of the present invention. For example, the translation of movement to a rotational mechanism as disclosed herein may be applicable to any medical device requiring the rotation of a distal tip upon actuation thereof, the medical device including, but not limited to cytology brushes, biopsy needles, snares, fluid sprayers, optic fibers, cutting tools, biopsy forceps, graspers, hooks and the like. Furthermore, although embodiments of the present invention are directed to a catheter sheet comprising an abutment and a needle having grooves, the present invention also covers embodiments wherein the abutments are formed on the needle and a groove is formed on the catheter shaft, as depicted in one embodiment in FIG. 5. Thus, it is to be understood that these embodiments have been described in an exemplary manner and are not intended to limit the scope of the invention which is intended to cover all modifications and variations of this invention that come within the scope of the appended claims and their equivalents.

What is claimed is:

1. A device for penetrating tissues within a living body, comprising:
a shaft extending from a proximal end to a distal end, the shaft comprising a channel extending therethrough from the proximal end to an opening in the distal end;
a needle extending within the channel, the needle comprising a tissue penetrating distal tip, a portion of an outer surface of the needle including a first structure extending along a path wrapping around a portion of a length of the needle, the first structure configured to mate with a second structure formed on a corresponding portion of an inner wall of the shaft, the first and second structures mating with one another so that, as the needle is translated axially through the channel, engagement between the first and second structures rotates the needle about an axis of the channel.

2. The device according to claim 1, wherein the first structure is a groove and the second structure is an abutment engaging the groove.

3. The device according to claim 2, wherein the groove is substantially helical

4. The device according to claim 3, wherein a length of the groove along the axis is less than a length of the needle along the axis.

5. The device according to claim 1, wherein the shaft defines a shaft lumen and the needle defines a needle lumen extending to a needle opening in a distal end of the needle, the needle lumen and shaft lumen being fluidly connected to one another.

6. The device according to claim 2, wherein the shaft is coupled to the needle by a hollow joint which fluidly couples a distal end of the shaft lumen to a proximal end of the needle lumen.

7. The device according to claim 6, wherein the joint allows the needle to rotate freely about the axis.

8. The device according to claim 2, wherein a length of the groove along the axis is selected to define a maximum length of the needle which may be extended from a distal end of the catheter

9. The device according to claim 8, wherein the length of the groove is selected to permit the needle to be fully retracted into the catheter.

10. The device according to claim 2, wherein the abutment is moveable between an engaged position in which the groove is engaged and a retracted position in which the needle is freed to move axially through the catheter without rotation about the axis.

11. The device according to claim 10, further comprising an actuator enabling a user to move the abutment between the retracted and engaged positions.

12. The device according to claim 10, wherein the abutment is a pawl rotatably coupled to an inner surface of the catheter.

13. The device according to claim 12, further comprising a biasing member coupled between the catheter and the pawl biasing the pawl toward the engaged position.

14. The device according to claim 12, wherein distal surfaces of the groove are ramped to enable the pawl to ratchet along an outer surface of the needle as the needle is withdrawn proximally into the catheter without rotating about the axis.

15. The device according to claim 12 wherein distal surfaces of the pawl are ramped to enable the pawl to ratchet along an outer surface of the needle as the needle is withdrawn proximally into the catheter without rotating about the axis.

16. The device according to claim 11, further comprising a mechanism moving the pawl to the retracted position when the needle has reached a distal most position and releasing the pawl to move back to the engaged position when the needle is fully retracted into the catheter.

17. The device according to claim 5, further comprising a proximal port fluidly coupled to the shaft lumen so that, negative fluid pressure applied thereto creates suction into the needle opening.

18. The device according to claim 2, wherein a pitch of the groove varies along a length of the groove.

19. The device according to claim 18, wherein the pitch of the groove is reduced at at least one of the proximal and distal ends thereof relative to a pitch of a central portion of the groove.

20. The device according to claim 1, further comprising an actuator, wherein a proximal end of the shaft is coupled to the actuator.

21. The device according to claim 1, wherein the a maximum outer diameter of the first helical portion exceeds an inner diameter of a portion of the channel distal of the second helical path to define a maximum extension of the tip from the distal opening of the channel.

22. The device according to claim 21, further comprising a plurality of surface incongruities extending along a third helical path along a portion of the needle member which, when the needle is extended distally from the distal opening of the channel, are located distal of the distal opening.

23. The device according to claim 22, wherein the surface incongruities include one of projections from an outer surface of the needle member, indentations in the outer surface of the needle member and insets in the outer surface of the needle member of a material having echo-sonic location properties different from a material of which surrounding portions of the needle member are formed.

24. The device according to claim 21, wherein the needle member includes a needle lumen extending through at least a portion there of from an opening in the distal tip.

25. The device according to claim 24, wherein an inner surface of the needle lumen is substantially smooth.

26. A method for taking tissue samples comprising:
inserting into a body to a location adjacent a target site, a device comprising a catheter slidably housing a needle and a shaft, a proximal end of the needle being rotatably coupled to a distal end of the shaft, the needle including a first structure extending along a path wrapping around portion of a length of the needle and engaging a second structure formed on a wall of the catheter; and
moving the shaft axially within the catheter to move the needle axially in and out of a distal end of the catheter into the target site, engagement of the first structure with the second structure rotating the needle about an axis of the needle.

27. The method of claim 26, further comprising the step of applying suction to a lumen extending through the shaft and the needle to a distal opening in the needle to draw tissue from the target site into the lumen.

28. The method of claim 26, further comprising the step of operating an actuator to move the first and second structures between an engaged position in which the first and second structures are in contact with one another to convert axial movement of the needle into rotational movement and a retracted position in which the first and second structures do not contact one another to permit translational movement of the needle relative to the shaft.

29. The method of claim 26, wherein the device is inserted to the target site through a body lumen.

30. The method of claim 29, wherein the device is inserted into the body lumen via a naturally occurring body orifice.
